(19) United States
(12) Patent Application Publication
(10) Pub. No.: US 2022/0015691 A1
(43) Pub. Date: Jan. 20, 2022

(54) VOICE TRAINING THERAPY APP SYSTEM AND METHOD

(71) Applicant: Steven Darroh, Austin, TX (US)

(72) Inventor: Steven Darroh, Austin, TX (US)

(21) Appl. No.: 17/253,898

(22) PCT Filed: Dec. 18, 2020
(86) PCT No.: PCT/US2020/065869

§ 371 Date: Dec. 18, 2020

Related U.S. Application Data
(60) Provisional application No. 62/949,455, filed on Dec. 18, 2019.

(51) Int. Cl. A61B 5/00; G09B 19/04; G16H 20/30

(52) U.S. Cl. CPC A61B 5/4803; G16H 20/30; G09B 19/04

(57) ABSTRACT

A method of providing voice therapy or training to a patient or other person over a communications network. The method includes delivering by a server computer communicatively connected to the communications network, a digital exercise instruction to a user client device communicatively connected to the communications network. The method includes receiving over the communications network from the user client device a digital voice signal representing an analog voice signal input to the user client device by the patient or other person, and storing the digital voice signal in a database communicatively connected to the server computer. The method also includes delivering a website to a Speech Language Pathologist (SLP) device or other administrator device communicatively connected to the communications network and providing access to the digital voice signal to the administrator device.

### CROSS-REFERENCE TO RELATED APPLICATIONS

The present application is a conversion and has benefit of priority of U.S. Provisional Patent Application No. 62/949,455, titled “A Digital Voice Therapy for Parkinson's Disease,” filed on Dec. 18, 2019, co-pending and having at least one same inventor of the present application.

### TECHNICAL FIELD

The invention generally relates to voice training devices, and more particularly relates to systems and methods for network communications and devices for administration of voice therapy.

### BACKGROUND

Voice therapy is critical for many people, including young and old. Conventionally, voice therapists have met face-to-face in person with patients for voice training. Growth of the Internet and computer communications has led to at least certain types of medical and physical administration as an acceptable form of therapy by patients.

A particular instance in which voice therapy may be required is by patients of Parkinson's Disease (PD). Patients with Parkinson's Disease suffer progressive degeneration of nerve cells in part of the brain called the substantia nigra, which controls muscle movements. The nerve cells lose the ability to produce an important chemical called dopamine. Symptoms of PD, which include degeneration of motor symptoms, generally develop slowly over years. As the disease worsens, non-motor symptoms become more common. The main motor symptoms are collectively called “parkinsonism.” The cause of PD is unknown, but it is believed to involve both genetic and environmental factors. There presently is no cure for PD. Non-pharmacological treatment, however, aims to improve the symptoms, i.e., the parkinsonism, through training and therapy. For purposes of this disclosure, the terms “therapy,” “training,” “exercise,” “coaching,” “treatment,” “administration,” and similar words, are intended to have similar and broadest meanings in respect of voice treatment or assistance, whether administered, coached or directed by a professional, such as a Speech Language Pathologist (SLP), a voice therapist, a voice trainer, or other person, whether assisted by predesigned procedures or processes of programs for computer and network devices, and/or otherwise.

Voice therapy is typically performed by voice therapists, such as SLPs, or similar persons, by coaching and training the patient to best voice sounds and motions creating those sounds. The therapy also teaches quality and volume of vocalization. Evidence-based research into voice therapy for patients with PD, for example, demonstrates that these patients can improve vocal volume and quality with exercise that trains them to speak more loudly. A person with PD may typically not realize that they are speaking quietly. Vocal exercises, for these and other persons, can be important to “recalibrate” voice and vocalization. Voice deficiencies can impact a person's communication professionally and personally. By practicing speaking more loudly, a person, such as a PD patient, can increase his/her typical voice effort that in turn produces a voice more audible to a listener. Other voice impediments can be treated, as well, by voice therapy.

More specifically, with respect to PD patients, current voice therapy and treatment options are limited. For instance, the Lee Silverman Voice Treatment (LSVT) is a one-on-one model that recommends a patient meet with a Speech Language Pathologist (SLP) four times a week for four consecutive weeks. Based on the patient's place of residence, the treatment can cost between about $2000 and about $4000 in a single month. Moreover, it is quite cumbersome for the patient to get to the SLP's office sixteen times in one month due to several limiting factors, such as location relative to an SLP, mobility impairments, doctors' appointments, professional career and others. For an SLP, current delivery treatments require that an SLP be certified through hours of training for providing voice therapy. Costs of certification can be as much as or over about $1,000 initially, and there are re-certification costs thereafter. These costs are then passed on to the patient/customer through higher prices for speech therapy services. Another one-on-one model of an existing treatment is the Parkinson Voice Project's SpeakOUT! This also requires a certified and trained SLP for administering therapy and treatment.

Conventional models of voice therapy, therefore, require an SLP to administer the therapy. As a voice specialist, the SLP provides prompts and feedback/cues to improve the patient's quality of voice and loudness. Treatment by SLPs for persons with PD can be extensive and long term, and the SLPs must be certified in the proprietary models. These typical voice therapies, therefore, are expensive and time consuming, and SLPs are in high demand.

It would, therefore, be a significant improvement in the art and technology to provide systems and methods for administration of voice therapy or training. It would also be a significant improvement to reduce the costs and requirements involved in administering voice therapies and training. It would, moreover, be an improvement to provide for easier and more facilitated access to SLPs and voice therapies and training, even for those less able to travel and be present. Furthermore, it would be beneficial to patients, such as, for example, PD patients, and others to provide effective voice treatment systems and methods that overcome the drawbacks and limitations of conventional activities and solutions.

### SUMMARY

An embodiment of the invention includes a system for speech therapy over a computer network. The system includes a server device communicatively connected to the computer network, the server device includes at least a processor and memory, a user client device communicatively connected to the computer network, the user client device includes at least a microphone and an analog-to-digital converter, an administrator device communicatively connected to the computer network, the administrator device includes at least a digital-to-analog converter, a speaker and an input device, and a database storage communicatively connected to the server device. The memory of the server device includes instructions for controlling the server device in mediating digital voice signals received from the user client device and digital exercise instructions from the administrator device, storing in the database the digital voice signals received from the user client device, serving a website portal of the server computer to the administrator device, the website portal allows the administrator device to retrieve the digital voice signals and to specify digital exercise instructions for the user client device, and delivering the digital exercise instructions to the user client device.

Another embodiment of the invention is a method of providing voice training to a patient over a communications network. The method includes delivering by a server computer communicatively connected to the communications network, a digital exercise instruction to a user client device communicatively connected to the communications network, receiving over the communications network from the user client device a digital voice signal representing an analog voice signal input to the user client device by the patient, storing the digital voice signal in a database communicatively connected to the server computer, delivering a website to an administrator device communicatively connected to the communications network, and providing access to the digital voice signal to the administrator device.

Yet another embodiment of the invention is a computer readable non-transitory medium having instructions for delivering over a computer network a digital exercise instruction to a user client device communicatively connected to the computer network, receiving over the computer network from the user client device a digital voice signal representing an analog voice signal input to the user client device by the patient, storing the digital voice signal in a database, delivering over the computer network a website to an administrator device communicatively connected to the computer network, and providing access over the computer network to the digital voice signal to the administrator device.

Another embodiment of the invention is a system for voice therapy and training over a communications network. The system includes a processor communicatively connected to the communications network, memory communicatively connected to the processor, an output device communicatively connected to the processor for delivering a voice exercise instruction, a microphone communicatively connected to the processor for receiving analog audio voice signals, a transducer communicatively connected to the microphone and the processor for converting the analog audio voice signals to analog electrical voice signals, and an analog-to-digital converter communicatively connected to the transducer and the processor for converting the analog electrical voice signals to digital voice signals.

Yet another embodiment of the invention is a system for voice therapy and training over a communications network. The system includes a processor communicatively connected to the communications network, memory communicatively connected to the processor, an input device communicatively connected to the processor for providing a voice exercise instruction, a digital-to-analog converter for converting a digital voice signal to analog voice signals, and a speaker communicatively connected to the processor and the digital-to-analog converter for outputting an analog audio voice signal in respect of the digital voice signal.

### BRIEF DESCRIPTION OF THE DRAWINGS

The present invention is illustrated by way of example and not limitation in the accompanying figures, in which like references indicate similar elements, and in which:

FIG. 1 illustrates a block diagram of an environment, according to certain embodiments of the invention;

FIG. 2 illustrates a graphical representation of typical volumes of audio levels and associated examples, according to certain embodiments of the invention;

FIG. 3 illustrates an exemplary schematic illustration of vocal intensity recorded over time, according to certain embodiments of the invention;

FIG. 4 illustrates a flow diagram illustrating a method to provide a digital voice treatment for people with PD, according to certain embodiments of the invention;

FIG. 5 illustrates a block diagram of a machine in the example form of a computer system within which instructions for causing the machine to perform any one or more of the methodologies discussed herein may be executed, according to certain embodiments of the invention;

FIG. 6 illustrates a system for voice exercise and therapy, according to certain embodiments of the invention;

FIG. 7 illustrates a method of a user client device for voice exercise and therapy, according to certain embodiments of the invention;

FIG. 8 illustrates a method of a server computer for voice exercise and therapy, according to certain embodiments of the invention; and

FIG. 9 illustrates a method of a Speech Language Pathologist (SLP) device for voice exercise and therapy, according to certain embodiments of the invention.

### DETAILED DESCRIPTION

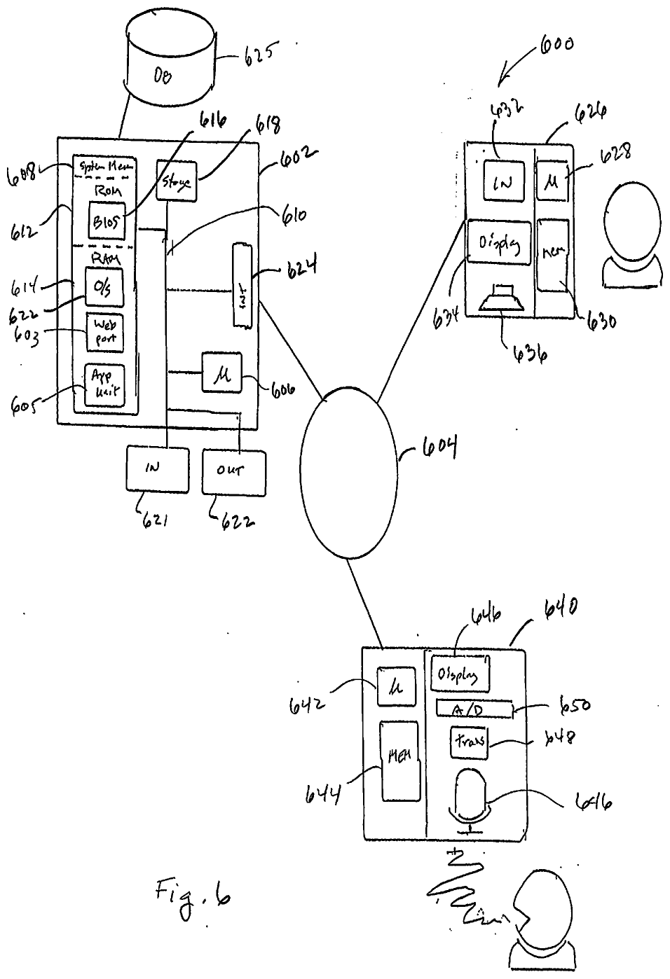

Referring to FIG. 6, a non-exclusive example embodiment of a system 600 includes a server computer 602 communicatively connected to a communications network 604. The server computer 602 processes a website portal unit 603 and an application unit 605 of or accessible to the server computer 602. The website portal unit 603 provides a website accessible on the network 604, and the application unit 605 provides a back office process in conjunction with voice therapy operations of the system 600.

The server computer 602 includes one or more computer systems including a processor 606, memory 608, and a system bus 610 that couples system components, including the memory 608, to the processor 606. The memory 608 may include a read only memory (ROM) 612 and a random access memory (RAM) 614. A basic input/output system (BIOS) 616 containing the basic routines that help to transfer information between elements within the computer system is stored in the ROM 612. The server computer 602 may also include a storage drive 618. The server computer 602 may also include input peripheral device 621 and output peripheral device 622. The storage drive 618 and the peripheral devices 621, 622 are connected to the system bus 610 by relevant interface. A number of modules can be stored in the memory 608 or storage drive 618, including an operating system 622, the website portal unit 603 and the application unit 605. The server computer 602 also includes a communication interface device 624 for receiving and sending information over the communications network 604.

The server computer 602 may, as non-exclusive example, be or include one or more server computer communicatively connected to the network 604 for processing software modules stored in memory, controlling interconnected hardware elements, and combinations of these, specially configured to provide operations and services later described. Although the server computer 602 is illustrated as a single device, the server computer 602 could be a distributed computing system comprising more than one server or computing device. The server computer 602 may, for non-exclusive example, be a cloud server.

An administrator device 626, such as a device of a Speech Language Pathologist (SLP) or other coach, therapist, or other, is communicatively connected to the communications network 604. The administrator device 626 is operable by a Speech Language Pathologist user to access the website portal unit 603 over the network 604, in conjunction with speech training or therapy to ascertain speech exercise progress of patients and historical and analytical data.

The administrator device 626 includes at least a processor 628 and memory 630. The administrator device 626 also includes a speaker 636 for delivering voice sounds corresponding to voice files to a Speech Language Pathologist operating the device 626. The administrator device 626 also includes an input device 632, for nonexclusive example, a keyboard, mouse, touch screen or other. Other peripherals and devices, such as a display 634 or other input or output device may be included in or communicatively connected to the administrator device 626. A system bus connects the memory 630, as well as the speaker 636, and any input device 632 and display 634 or other input or output device, to the processor 628. The administrator device 626 also includes a communication interface device (not shown in detail) for sending and receiving information over the communications network 604.

The administrator device 626 may, as non-exclusive example, be or include one or more processor or computer communicatively connected to the network 604 for processing software modules stored in memory, controlling interconnected hardware elements, and combinations of these, specially configured to provide operations and services later described. Although the administrator device 626 is illustrated as a unitary device, the administrator device 626 could be communicatively connected hardware, software, other devices, and combinations.

A user client device 640 is communicatively connected to the communications network 604. The user client device 640 is operable by a speech user client to access the application unit 605 over the network 604, in conjunction with a speech therapy or training exercise.

The user client device 640 includes at least a processor 642 and memory 644. The user client device 640 also includes a microphone 646 for receiving analog voice signals from a client. The user client device 640 also includes a display device 646 for presenting textual or other visual voice exercises to the client operating the device 640. Other peripherals and devices, such as other input or output device may be included in or communicatively connected to the user client device 640. A system bus (not shown in detail) connects the memory 642, as well as the microphone 646, the display 634, and any other input or output device, to the processor 648. The user client device 640 also includes a communication interface device (not shown in detail) for sending and receiving information over the communications network 604.

The user client device 640 further includes a transducer 648 and an analog to digital (A/D) converter 650. In non-exclusive examples, the transducer 648 and A/D converter 650 are implemented in hardware, software or combinations, in the user client device 640, which may be or include for example, a digital signal processor (DSP), an application specific integrated circuit (ASIC), the processor 648, amplifier, and other devices and combinations. The user client device 640 operates to receive through the microphone 646 analog voice signals of the client and convert these to digital voice files that are communicated by the user client device 640 to the application unit 605.

The user client device 640 may, as non-exclusive example, be or include one or more processor or computer communicatively connected to the network 604 for processing software modules stored in memory, controlling interconnected hardware elements, and combinations of these, specially configured to provide operations and services later described. Although the user client device 640 is illustrated as a unitary device, the user client device 640 could be communicatively connected hardware, software, other devices, and combinations.

A database 625 is included in or communicatively connected to the server computer 602. The database 625 is implemented as hardware, software or combinations. An example of the database 625 is a relational database, spreadsheet, or other database. The database 625 stores digital information representing textual or other visual voice exercises for delivery by the application unit 605 of the server computer 604 to the user client device 640. When a client user of the user client device 640 performs speech therapy or training exercises by speaking into the microphone 646 of the user client device 640, the application unit 605 receives digital files of the client's analog voice from the user client device 640 over the network 604. The database 625 also stores and makes available to the website portal unit 603 digital files representing the analog voice signals of the client responsive to a client performing speech therapy or training exercise. The administrator device 626 accesses a website of the website portal unit 603 over the network 604, to receive the digital files of voice signals, retrieve historical, analytical, and other information of the database 625 relevant to a client and voice exercises. New, modified, substitute or additional speech therapy or training exercises, as directed by a Speech Language Pathologist or other via the administrator device 626, may be stored in the database 625, for non-exclusive example, as communicated to the server computer 604 by the administrator device 626 in the website of the website portal unit 603.

The network 604 may, as non-exclusive example, be or include any one or more telecommunications and/or data network(s), or combination of such networks, whether public, private or combinations of these, including, for example, the Internet, a local area network, wide area network, intranet, public switched telephone network (PSTN), wireless (e.g., cellular, WiFi, WLAN, GPS, infrared, satellite, radio frequency, or other) network, satellite network, other wired or wireless communication link or channel, combination of links or channels, or any combination of these. A non-exclusive example of the communications network 604 is or includes the Internet, including but not limited to any and every possible combination of a wired data link, wireless cellular data link, and other link connected to the Internet (e.g., connected directly or indirectly connected through other links or networks).

In operation, the system 600 makes speech training exercises available to clients operating user client device(s) 640 and allows the speech language therapist, trainer, coach or other administrator, operating the administrator device 626 to access and assess the voice of the clients through digital communications over the network 604. The server computer 602 communicates to the user client device 640 a text or visual exercise for training the client user's voice. The client, for nonexclusive example, a speech patient, responds with analog voice signals to the user client device 640. The user client device 640 converts the analog voice signals to digital files representing the analog voice signals. The user client device 640 communicates the digital files representing analog voice signals to the server computer 604.

The server computer 604 stores the digital files representing analog voice signals in the database 625. The administrator device 626 can, through a website of the server computer 604 accessible over the network 604, access the digital files representing analog voice signals from the user client device 640. The administrator device 626 converts the digital files back to analog voice signals output by the administrator device 626 to the Speech Language Pathologist. The Speech Language Pathologist can also, via the administrator device 626, communicate through the website any next, new, additional or substitute speech therapy or training exercise as a digital file accessible by the user client device 640.

In non-exclusive embodiments, the database 625 may contain one or more initial speech therapy or training exercise for the user client device 640. The user client device 640 may receive exercises at any desired time or increment, according to the desired implementation. Exercises may be communicated to the user client device 640 through an application program (App), web browser or other communication vehicle of the user client device 640, according to the desired implementation. If an App is processed by the user client device 640 in embodiments, the App can provide the desired functionality of receiving and displaying the exercise as visible text or otherwise, capturing analog voice signals of the patient or other client responsive to exercise instructions, converting these analog voice signals to digital files representing the voice signals, and communicating the digital files over the network 604 to the server computer 604.

Further in embodiments, the administrator device 626 may access over the network 604 a website of the server computer 602. The website may allow the administrator device 626 to receive over the network 604 the digital files representing analog voice signals of the patient or other client (i.e., those of the user client device 640 responsive to exercises). The website may also allow the administrator device 626 to view historical, analytical and other information in respect of speech therapy or training patients or clients using user client device(s) 640 to perform voice exercises. Further, the administrator device 626 may through the website and over the network 604 add new speech therapy or training exercises for patients or other clients, prescribe particular exercise(s) for respective patients or clients, and otherwise modify, substitute and implement exercises and exercise programs for patients or clients.

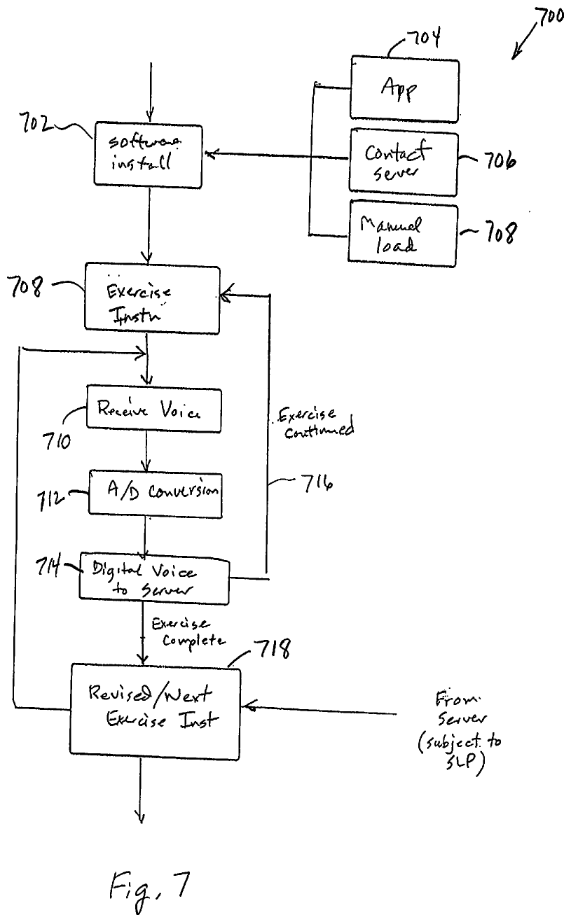

Referring to FIG. 7, a method 700 of operation of a user client device includes installing software 702. The software may be installed 702 to the user client device by communicating 704 with a network resource server, such as, for example, Google Play Store, Apple App Store, or otherwise, if an App is employed by the user client device. Alternately, a server computer may be accessed 706 as source for the software, if available per the embodiment and implementation. The software could in other alternatives be manually or otherwise loaded on the user client device.

Once the software is installed 702, the client user of the user client device can commence processing exercise instructions 708 by the user client device. It is contemplated that initial exercise instructions may be useful to the Speech Language Pathologist to benchmark the client's voice capabilities, such as, for non-exclusive example, tone, volume and other characteristics.

Responsive to processing exercise instructions 708, the client can provide analog voice signals received 710 by the user client device. The user client device converts 712 the analog voice signals to digital data representing the analog voice signals. The digital data is delivered 714 over the network by the user client device to the server computer. In a step 716, processing exercise instructions 708 continues.

If exercise is completed by the client in the user client device, the user client device can then, or at other time or manner, receive further exercise instructions 718 which may be next, new, revised, modified, additional, substitute or other instructions as received from the server computer (i.e., the administrator device may provide over the network to the server computer the further exercise instructions, as may be applicable). Upon receiving further exercise instructions 718 by the user client device, the method 700 returns to receiving 710 analog voice signals of the client responsive to the exercise instructions.

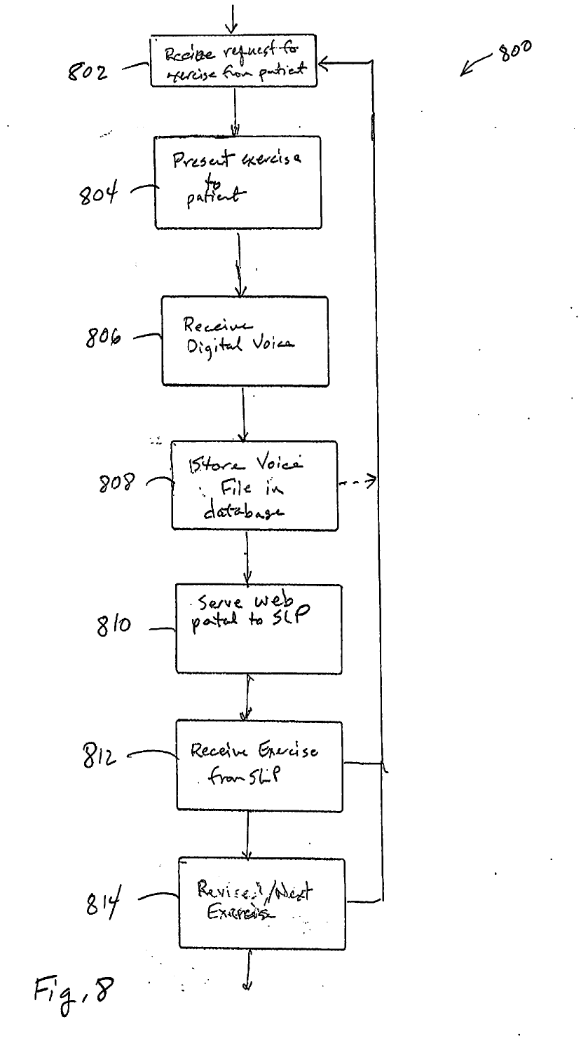

Referring to FIG. 8, a method 800 of operation of a server computer includes receiving a request over the network from the user client device for an exercise instruction for processing 708 (shown in FIG. 7) by the user client device. The server computer delivers 804 over the network the exercise instruction to the user client device.

Responsive to delivering 804, the server computer receives 806 from the network (i.e., from the user client device) digital voice file(s) representing the analog voice signal of the client. The digital voice files are stored 808 by the server computer in the database. Thereafter the method 800 may return to receiving 802 request for exercise instructions from the user client device, or else the method 800 may continue.

If the method 800 continues, a Speech Language Pathologist, other speech therapist or other administrator operating the administrator device accesses over the network a website portal of the server computer available over the network. The server computer delivers 810 the website to the administrator device. The administrator device may then transmit over the network to the server computer further exercise instructions. These further exercise instructions are received 812 by the server computer from the network.

The method 800 of the server computer may thereafter return to receiving 802 request for exercises from the user client device over the network. Alternately or additionally, the method 800 continues with the server computer receiving 814 on the network additional, new, next, modified, supplemental, substitute or other exercise instructions, from the administrator device, or as otherwise implemented in the embodiment. After receiving 814, the server computer continues receiving 802 request from the user client device.

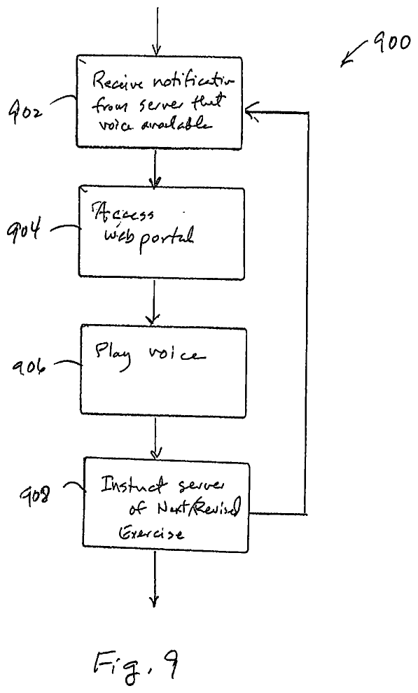

Referring to FIG. 9, a method 900 of operation of an administrator device includes receiving notification 902 from a server computer that digital voice files (i.e., representing analog voice signals of a client user of a user client device) are available to the administrator device. The administrator device accesses 904 over the network the website portal of the server computer. The website portal may provide to the administrator device a particular interface of menu, options, settings, and so forth, which may include various options for receiving historical, analytical or other information of clients and voice signals.

The administrator device can select to receive, stream or otherwise play 906 analog voice signals (i.e., represented by digital files of the database) from the server computer. In playing 906 analog voice signals, the administrator device performs digital to analog conversion of the digital files and outputs analog voice signals through an applicable interface, such as, for example, a speaker. Responsive to playing 907 analog voice signals by the administrator device, the administrator device may (e.g., if input or otherwise provided or directed by a Speech Language Pathologist based on the analog voice signals) deliver 908 over the network to the server computer further instructions, modification or additions or substitutions to instructions, or otherwise communicate with the server computer. The method 900 of the administrator device then returns to the receiving notification step 902.

### NON-EXCLUSIVE EXAMPLE

A non-exclusive example of certain of the embodiments follows. The example relates to methods and systems to provide a digital voice training for people with Parkinson's Disease (PD). The digital voice treatment aims to strengthen their voice and restore quality of life through improved communication. The following details are intended to provide non-exclusive example implementations to one of ordinary skill in the art and not as limitation to the example.

As used herein, a computing device near a user is referred to as a “first computing device”. As also used herein, a computing device near a Speech Language Pathologist is herein referred to as a “second computing device”.

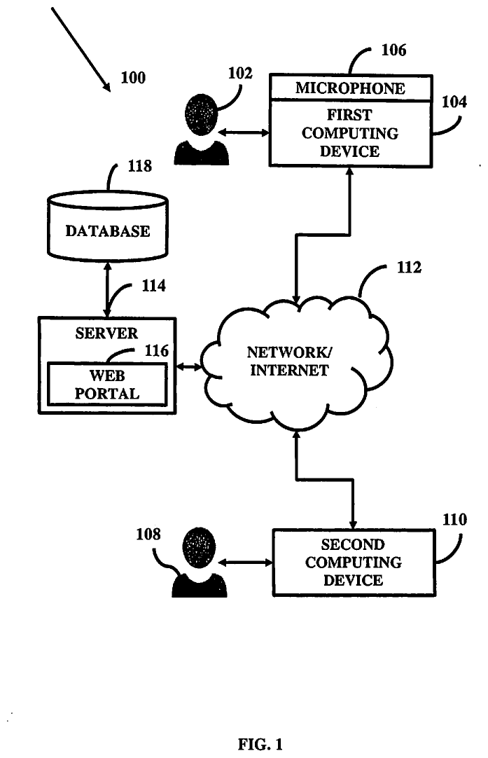

FIG. 1 is a block diagram of an environment, according to the embodiments as disclosed herein. The environment 100 includes a user 102, a first computing device 104, a microphone 106, a Speech Language Pathologist 108, a second computing device 110, a network 112, a server 114, a web portal 116 and a database 118.

The user 102 is a person with a hypophonic voice. Hypophonia is frequently caused by neurological disorders or acquired brain injuries (ABIs). Examples of the neurological disorders and ABIs include, but are not limited to brain tumors, traumatic brain injuries, Parkinson's Disease (PD), and stroke. The method described herein is performed fora person with PD. However, it is to be noted that the method may be performed for persons with similar or other voice symptoms.

Although PD presents differently for different persons, four hallmark symptoms are tremor, rigidity, bradykinesia, and loss of balance. Typically the user 102 with PD may have trouble moving or speaking. Problems with memory, senses or mood may also arise. Many people with PD experience changes in their voice or speech. The voice may get softer, breathy or hoarse that causes difficulty for others to understand what is said. Speech may also be slurred.

The first computing device 104 is a portable electronic or a desktop device operated by the user 102. Further, the first computing device 104 is configured with the in-built microphone 106. Examples of the first computing device 104 include, but are not limited, to a personal computer (PC), a mobile phone, a tablet device, a personal digital assistant (PDA), a smart phone, a laptop and pagers. In some embodiments, the computing device includes a microphone, a loud speaker, a web cam and a sound pressure level meter attached thereto.

The microphone 106 is a type of a transducer that captures audio by converting sound waves (acoustical energy) into electrical signals (the audio signal). Further, the microphone 106 is in-built and mostly located on the back of the phone near the bottom of the handset. It is to be noted that the microphone may be located at any other appropriate location in the device. Specifically, the microphone 106 is used as a sound level meter to assess noise or sound levels by measuring sound pressure of the user's voice.

The Speech Language Pathologist (SLP) 108 is a highly-trained professional who evaluates and treats people who have difficulty with speech or language. Further, the Speech Language Pathologist 108 evaluates, diagnoses and treats speech, language, communication and swallowing disorders. A Speech Language Pathologist, at a minimum, holds a master's degree in Communication Sciences and Disorders (CSD) and a certification for the said. The method described herein allows a Speech Language Pathologist without additional certification to provide voice training or treatments to the user 102.

The second computing device 110 is a portable electronic or a desktop device operated by the Speech Language Pathologist 108. Examples of the second computing device 110 are similar to the aforementioned first computing device 102.

It must be noted that the first computing device 102 and the second computing device 110 are configured with a user interface (not shown in FIG. 1). Examples of the user interface include, but are not limited to display screen, touch screen, keyboard, mouse, light pen, appearance of desktop, illuminated characters and help messages. The user interface displays prompts and a vertical sound bar that visually illustrates the loudness of the user's 102 voice.

It is to be noted that the user 102 and the Speech Language Pathologist 110 may or may not be located at different geolocations.

The first computing device 104 is attached through the network 112, such as the Internet to the second computing device 110 near the Speech Language Pathologist 108. In some embodiments, the Speech Language Pathologist's computing device 110 may also have a web cam and a loudspeaker attached thereto.

Examples of the network 112 include, but are not limited to, wireless network, wire line network, public network such as the Internet, Intranet, private network, General Packet Radio Network (GPRS), Local Area Network (LAN), Wide Area Network (WAN), Metropolitan Area Network (MAN), cellular network, Public Switched Telephone Network (PSTN), personal area network, and the like. The network 112 may be operable with cellular networks, Bluetooth network, Wi-Fi networks, or any other networks or combination thereof.

The first computing device 104 and the second computing device 110 are configured with a non-transitory computer-readable medium (an application program), the contents of which cause it to perform the method disclosed herein.

The server 114 hosts and runs the web portal 116. Web pages are distributed as they are requisitioned through the server 114. The basic objective of the server 114 is to process and deliver web pages.

The web portal 116 may be viewable with a standard web browser, such as Internet Explorer®, Firefox®, Mozilla®, Safari®, Chrome® and/or other browser or device. The web portal 116 generates and transmits an initial page to the SLP 108. Further, the web portal 116 integrates with the app downloaded in the first computing device 102. It gathers information such as user profile, health reports and assignments given by the SLP 108. The web portal 116 collaborates the said information from one or more users and presents it to the SLP 108. Accordingly, the second computing device 110 connects to the web portal 116.

The database 118 is responsible for storing all the information communicated between the user 102 and the Speech Language Pathologist 108. Further, the database 118 keeps track of measurements and other indicia of every task/exercise assigned to the user 102 and the user's progress throughout the voice treatment.

To begin, the app is downloaded in the first computing device 102. The SLP 108 accesses a website using the second computing device 110. The website typically allows the SLP 108 to manage assignments of the user 102, specifically through a web portal which synchronizes to the app downloaded in the first computing device 102. In another embodiment, the SLP 108 manages assignments of the user 102 through an app downloaded to the second computing device 110.

The Speech Language Pathologist 108 instructs the user 102 to perform a task. It is to be noted that the SLP may not be present while the user is using the app. In such a scenario, the instructions from the SLP and “homework” assignment may be assigned to the user asynchronously. The SLP may check the user's progress later in future.

The task (as mentioned above) is vocalization of a word, phrase, or sentences, for instance sustaining “ah,” counting 1-10, or verbalizing response to prompts, elicited by a plurality of speaking prompts. As the user 102 performs the requested task, the microphone 106 captures the loudness of the voice and displays as a vertical sound bar on the second computing device 110. Based on the measurements illustrated in the sound bar, the App trains the user 102 to increase their volume. The training is accomplished through multiple sessions. The sessions are approximately 10-20 minutes, or as otherwise implemented as desired. It is to be noted that the sessions may vary with reference to its time duration. Further, real-time feedback is provided to the user 102 on the loudness of his/her voice through the visual sound bar. As a result, the user 102 would learn the effort required to audibly project his or her voice and carry over that effort into his or her typical everyday interactions.

It should be appreciated by those of ordinary skill in the art that FIG.1 depicts the computing device in an oversimplified manner and a practical embodiment may include additional components and suitably configured processing logic to support known or conventional operating features that are not described in detail herein.

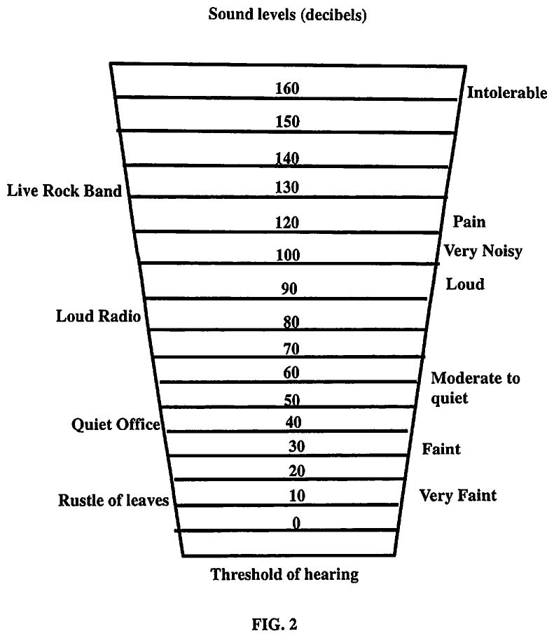

FIG. 2 is a schematic representation of typical volumes of audio levels and associated examples, according to the embodiments as disclosed herein.

The volumes of audio levels are categorized based on the threshold of hearing. For instance, a 10 dB volume of audio is very faint like the rustle of leaves. Similarly, a 130 dB volume of audio is painful and can be heard at a live rock concert.

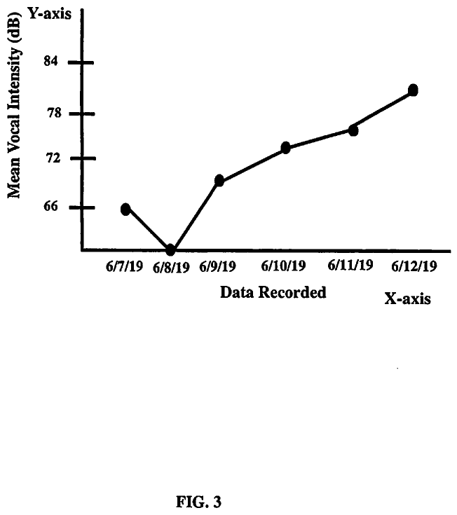

FIG. 3 is an exemplary schematic illustration of vocal intensity recorded over time, according to the embodiments as disclosed herein.

The graph illustrates the data recorded over time on the X-axis and the mean vocal intensity in the Y-axis. The loudness of the voice may vary from below 68 dBa (generally too soft), 72-78 dBa (generally appropriate speaking levels), to over 82 dBa (generally too loud).

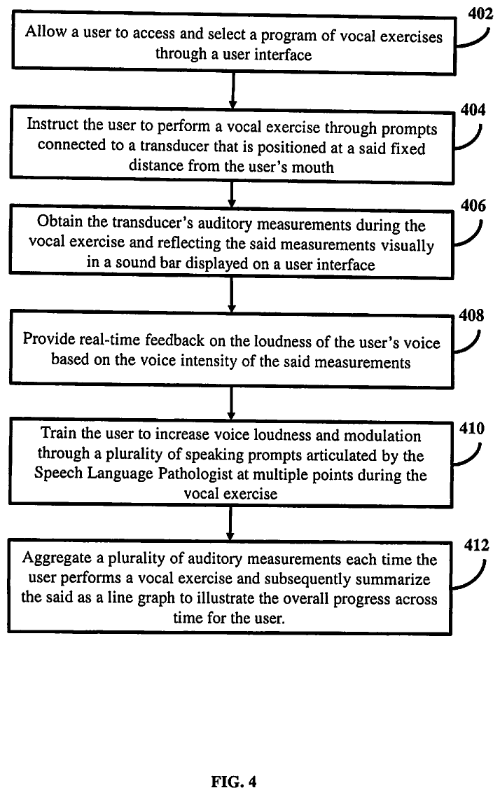

FIG. 4 is a flow diagram illustrating a method to provide a digital voice treatment for people with PD, according to the embodiments as disclosed herein. The method begins at step 402.

The method described herein associates two types of people namely the person with PD (can also be referred herein as “user” or “patient” or “client”) and the Speech Language Pathologist (SLP). The patient may be in his/her early, mid-stages or late stages of PD.

At step 402, a user is allowed to access a schedule of vocal exercises through a user interface.

The schedule described herein is a software-led therapy for people with PD instead of a one-to-one therapy, though the said is to be used under direction of an SLP or other administrator. The schedule is selected by the user who is located remotely. In some embodiments, the user's Speech Language Pathologist may select the schedule and then assign it to the user. In such a scenario, the Speech Language Pathologist can check the user's progress later on in future.

Accordingly, the user can use the app independently.

At step 404, a user is instructed to perform the vocal exercises through prompts connected to a transducer that is positioned at a said fixed distance from the user's mouth.

The user with PD is near the computing device described in FIG. 1. Specifically, the computing device may be a personal computer or a phone for the purposes of the method described herein. Typically, the transducer (microphone) is placed approximately 12-20 inches from the user's mouth. In some embodiments, the transducer may be adjusted to a suitable or convenient distance from the user's mouth such that the audio is captured.

To begin with, the app prompts the user to perform a task. The task is typically vocalization (the process of producing sounds with the voice) of one or more words. After the instructions are passed to the user, the user performs the task (for instance, say “oh” and hold for 10 seconds, completing a common phrase, reading aloud a joke or answering trivia questions, or otherwise as implemented).

At step 406, the transducer's auditory measurements are obtained during the vocal exercise and the said measurements are then reflected visually in a sound bar displayed on the user interface.

The method described herein utilizes the transducer, specifically a microphone in the phone or tablet as a sound level meter. In some embodiments, the user may buy a sophisticated microphone and attach it to his/her phone or computer to more precisely capture the vocal intensity compared to mics in phones or computers. The sound level meter is a handheld instrument with the microphone that is used for acoustic (sound that travels through air) measurements. Sound pressure is measured by a device often referred to as a sound pressure level (SPL) meter, decibel (dB) meter, noise meter or noise dosimeter. The sound is then evaluated within the sound level meter and the acoustic measurement values are shown on the display of the sound level meter.

Subsequently, the said acoustic measurement values are visually displayed on the user interface (i.e., screen of the phone or computer) to provide visual feedback for a user to visualize the volume at which he/she is speaking. It is to be noted that the Speech Language Pathologist may or may not be present before his/her computer while the user performs the exercises. This is illustrated through a vertical sound bar displayed on the user interface.

At step 408, based on the vocal intensity from the said measurements, real-time feedback on the loudness of the user's voice is provided.

The feedback is an ongoing process that happens throughout the program and any exercises or warm-up that is instructed. Typically, one or several sessions are assigned to the patient, in particular by an SLP, or the patient trains according to predetermined exercise of the app, or otherwise. These sessions are designed to recalibrate the patient's voice. In some embodiments, these sessions may be approximately 10-20 minutes. Further, the sessions may vary from warm-ups, exercises and homework. The sessions are collectively referred herein as app. The app leads the patient through vocal exercises practicing increased loudness, pitch change and volume change

Consequently, the program directs the patient to everyday speech exercises to recalibrate their voice. This section includes various exercises and levels of difficulty to keep the process lively and engaging.

Further, the user's exercises are tracked and updated every day. The patient and anyone of their choosing can access the data and track the progress over the weeks and months.

At step 410, the user is trained to increase voice loudness through a plurality of speaking prompts uploaded by a Speech Language Pathologist at various points during the program.

In one embodiment, the app may have default prompts written by an SLP. In another embodiment, the user's own SLP can override the defaults with prompts of their own.

The method delivers video modeling for proper warm-up technique complete with audio. Additionally, there are hundreds of voice prompts available. Examples of voice prompts include, but are not limited to tongue twisters, famous passage readings and jokes that keep the process engaging and entertaining throughout.

The method described herein is designed to aid the user to recalibrate his or her voice to speak more audibly. In some embodiments, the method may serve several cross benefits, for instance swallowing issues, strength training and so on. In such circumstances, an in-built web cam may be required.

It is to be noted that the user who performs vocal exercises to improve their voice quality may also have their swallow positively affected as well.

The SLP can customize the schedule for the user and check the user's progress and completion. This happens asynchronously between the SLP and the user.

Additionally, the SLP can assign homework digitally to the user and check his/her progress later in the future. Consequently, users who cannot frequently login to the app can still receive access to the voice treatment as the user's schedule allows.

At step 412, a plurality of auditory measurements is aggregated each time the user performs a vocal exercise and subsequently summarizes the said as a line graph to illustrate the overall progress across time for the user.

The microphone's auditory measurements are aggregated and recorded every time the user signs in to the app. The auditory measurements are then summarized in a single datum point that gets displayed on a line graph to show the overall progress across time for the user. In some embodiments, the line graph may be replaced to illustrate more granular access of the measurements.

The method ends at step 412.

The methods and systems described herein are beneficial for several reasons such as for non-exclusive example:
1. It offers superior access and affordability to voice treatment for people with PD and their Speech Language Pathologists.
2. It dramatically reduces the cost to users.
3. It allows for customization by participating Speech Language Pathologists.
4. It does not require proprietary certification of Speech Language Pathologists.
5. It improves access to voice training for any users that live remotely, have mobility impairments or otherwise are prevented from traveling to meet the Speech Language Pathologist frequently.
6. It exists digitally as opposed to physically in booklets.
7. It allows for continued voice training in the event that face-to-face contact is not advisable or allowable by state or other order or otherwise as a desired practice.

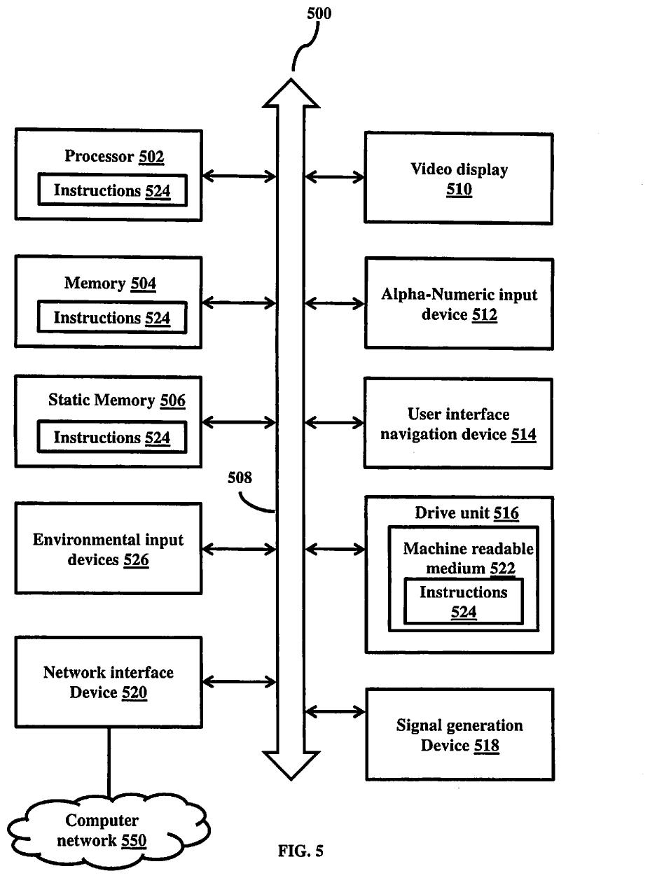

FIG. 5 is a block diagram of a machine in the example form of a computer system within which instructions for causing the machine to perform any one or more of the methodologies discussed herein may be executed. In alternative embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a Personal Digital Assistant (PDA), cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 500 includes a processor 502 (e.g., a central processing unit (CPU), a graphics processing unit (GPU), or both), a main memory 504, and a static memory 506, which communicate with each other via a bus 508. The computer system 500 may further include a video display unit 510 (e.g., a liquid crystal display (LCD) or a cathode ray tube (CRT)). The computer system 500 also includes an alpha-numeric input device 512 (e.g., a keyboard), a user interface (UI) navigation device 514 (e.g., a mouse), a disk drive unit 516, a signal generation device 518 (e.g., a speaker), and a network interface device 520. The computer system 500 may also include an environmental input device 526 that may provide a number of inputs describing the environment in which the computer system 500 or another device exists, including, but not limited to, any of a Global Positioning Sensing (GPS) receiver, a temperature sensor, a light sensor, a still photo or video camera, an audio sensor (e.g., a microphone), a velocity sensor, a gyroscope, an accelerometer, and a compass.

Machine-Readable Medium:

The disk drive unit 516 includes a machine-readable medium 522 on which is stored one or more sets of data structures and instructions 524 (e.g., software) embodying or utilized by any one or more of the methodologies or functions described herein. The instructions 524 may also reside, completely or at least partially, within the main memory 504 and/or within the processor 502 during execution thereof by the computer system 500, the main memory 504 and the processor 502 also constituting machine-readable media.

While the machine-readable medium 522 is shown in an example embodiment to be a single medium, the term “machine-readable medium” may include a single medium or multiple media (e.g., a centralized or distributed database, and/or associated caches and servers) that store the one or more instructions 524 or data structures. The term “non-transitory machine-readable medium” shall also be taken to include any tangible medium that is capable of storing, encoding, or carrying instructions for execution by the machine and that cause the machine to perform any one or more of the methodologies of the present subject matter, or that is capable of storing, encoding, or carrying data structures utilized by or associated with such instructions. The term “non-transitory machine-readable medium” shall accordingly be taken to include, but not be limited to, solid-state memories, and optical and magnetic media. Specific examples of non-transitory machine-readable media include, but are not limited to, non-volatile memory, including by way of example, semiconductor memory devices (e.g., Erasable Programmable Read-Only Memory (EPROM), Electrically Erasable Programmable Read-Only Memory (EEPROM), and flash memory devices), magnetic disks such as internal hard disks and removable disks, magneto-optical disks, and CD-ROM and DVD-ROM disks.

Transmission Medium:

The instructions 524 may further be transmitted or received over a computer network 550 using a transmission medium. The instructions 524 may be transmitted using the network interface device 520 and any one of a number of well-known transfer protocols (e.g., HTTP). Examples of communication networks include a local area network (LAN), a wide area network (WAN), the Internet, mobile telephone networks, Plain Old Telephone Service (POTS) networks, and wireless data networks (e.g., WiFi and WiMAX networks). The term “transmission medium” shall be taken to include any intangible medium that is capable of storing, encoding, or carrying instructions for execution by the machine, and includes digital or analog communications signals or other intangible media to facilitate communication of such software.

As described herein, computer software products can be written in any of various suitable programming languages, such as C, C++, C#, Pascal, Fortran, Perl, Matlab (from MathWorks), SAS, SPSS, JavaScript, AJAX, Java, Swift, Flutter, Objective C, or other. The computer software product can be an independent application with data input and data display modules. Alternatively, the computer software products can be classes that can be instantiated as distributed objects. The computer software products can also be component software, for example, Java Beans or Enterprise Java Beans. Much functionality described herein can be implemented in computer software, computer hardware, or a combination.

Furthermore, a computer that is running the previously mentioned computer software can be connected to a network and can interface to other computers using the network. The network can be an intranet, internet, or the Internet, among others. The network can be a wired network (for example, using copper), telephone network, packet network, an optical network (for example, using optical fiber), or a wireless network, or a combination of such networks. For example, data and other information can be passed between the computer and components (or steps) of a system using a wireless network based on a protocol, for example Wi-Fi (e.g., IEEE standard 802.11 including its sub-standards a, b, e, g, h, i, n, or other). In one example, signals from the computer can be transferred, at least in part, wirelessly to components or other computers.

It is to be understood that although various components are illustrated herein as separate entities, each illustrated component represents a collection of functionalities which can be implemented as software, hardware, firmware or any combination of these. Where a component is implemented as software, it can be implemented as a standalone program, but can also be implemented in other ways, for example as part of a larger program, as a plurality of separate programs, as a kernel loadable module, as one or more device drivers or as one or more statically or dynamically linked libraries.

In the foregoing, the invention has been described with reference to specific embodiments. One of ordinary skill in the art will appreciate, however, that various modifications, substitutions, deletions, and additions can be made without departing from the scope of the invention. Accordingly, the specification and figures are to be regarded in an illustrative rather than a restrictive sense, and all such modifications substitutions, deletions, and additions are intended to be included within the scope of the invention. Any benefits, advantages, or solutions to problems that may have been described above with regard to specific embodiments, as well as device(s), connection(s), step(s) and element(s) that may cause any benefit, advantage, or solution to occur or become more pronounced, are not to be construed as a critical, required, or essential feature or element.

What is claimed is:

1. A system for speech training over a computer network, comprising:
a server device communicatively connected to the computer network, the server device includes at least a processor and memory;
a user client device communicatively connected to the computer network, the user client device includes at least a microphone and an analog-to-digital converter;
an administrator device communicatively connected to the computer network, the administrator device includes at least a digital-to-analog converter, a speaker and an input device;
a database storage communicatively connected to the server device;
the memory of the server device includes instructions for controlling the server device in:
mediating digital voice signals received from the user client device and digital exercise instructions from the administrator device;
storing in the database the digital voice signals received from the user client device;
serving a website portal of the server computer to the administrator device, the website portal allows the administrator device to retrieve the digital voice signals and to specify digital exercise instructions for the user client device; and
delivering the digital exercise instructions to the user client device.

2. A method of providing voice training to a client over a communications network, comprising:
delivering by a server computer communicatively connected to the communications network, a digital exercise instruction to a user client device communicatively connected to the communications network;
receiving over the communications network from the user client device a digital voice signal representing an analog voice signal input to the user client device by the client;
storing the digital voice signal in a database communicatively connected to the server computer;
delivering a website to an administrator device communicatively connected to the communications network; and
providing access to the digital voice signal to the administrator device.

3. A computer readable non-transitory medium, comprising instructions for:
delivering over a computer network a digital exercise instruction to a user client device communicatively connected to the computer network;
receiving over the computer network from the user client device a digital voice signal representing an analog voice signal input to the user client device by the patient;
storing the digital voice signal in a database;
delivering over the computer network a website to an administrator device communicatively connected to the computer network; and
providing access over the computer network to the digital voice signal to the administrator device.

4. A system for voice training over a communications network, comprising:
a processor communicatively connected to the communications network;
memory communicatively connected to the processor;
an output device communicatively connected to the processor for delivering a voice exercise instruction;
a microphone communicatively connected to the processor for receiving analog audio voice signals;
a transducer communicatively connected to the microphone and the processor for converting the analog audio voice signals to analog electrical voice signals; and
an analog-to-digital converter communicatively connected to the transducer and the processor for converting the analog electrical voice signals to digital voice signals.

5. A system for voice training over a communications network, comprising:
a processor communicatively connected to the communications network;
memory communicatively connected to the processor;
an input device communicatively connected to the processor for providing a voice exercise instruction;
a digital-to-analog converter for converting a digital voice signal to analog voice signals; and
a speaker communicatively connected to the processor and the digital-to-analog converter for outputting an analog audio voice signal in respect of the digital voice signal.
